(19) United States
(12) Patent Application Publication
(10) Pub. No.: US 2011/0152979 A1
(43) Pub. Date: Jun. 23, 2011

(54) Microbe Reduction with Light Radiation

(75) Inventors: Tim Driscoll, Holyoke, MA (US); Wolfgang Neuberger, Dubai (AE)

(73) Assignee: CeramOptec Industries Inc.

(21) Appl. No.: 12/643,547

(22) Filed: Dec. 21, 2009

(51) Int. Cl. A61N 5/06

(52) U.S. Cl. 607/93; 607/90

(57) ABSTRACT

Present invention discloses methods and devices for treating skin and nail infections such as onychomycosis and dermatophytosis, caused by pathogenic microbes such as fungi. in a preferred embodiment, a method for treating skin and nail infections comprises irradiating the treatment site with a light radiation source operating at one or more near infrared wavelengths which are absorbed by the pathogenic microbes, specifically operating at 980±30 nm, 1470±40 nm and/or 1900±60 nm. In another preferred embodiment, a method for treating skin and nail fungus comprises irradiating the treatment site with a device emitting laser radiation of 980±30 nm wavelength. In another embodiment, a device for treating toenail fungus comprises a laser radiation source which emits a combination of radiation wavelengths at about 980±30 nm and 1470±40 nm; or 980±30 nm and 1900±60 nm; or 1470±40 nm and 1900±60 nm; or 980±30 nm, 1470±40 nm and 1900±60 nm. In another embodiment, devices for treating skin and nail infections include a laser radiation source and delivery apparatuses such as brush or toothed comb for nail and scalp infections respectively.

### BACKGROUND OF THE INVENTION

1. Field of the invention

The present invention relates to the use of light radiation to kill the microbes involved in a number of skin and nail microbial infections, preferably fungi causing dermatophytosis such as Tinea barbae, Tinea capitis, Tinea cruris and Tinea unguium.

2. Information Disclosure Statement

Dermatophytosis (tinea) infections are fungal infections caused by dermatophytes—a group of fungi that invade and grow in dead keratin. Several species commonly invade human keratin and these belong to the Epidermophyton, Microsporum and Trichophyton genera such as Epidermophyton floccosum, Microsporum canis, Microsporum audouinii, Trichophyton interdigitale/mentagrophytes, Trichophyton tonsurans, Trichophyton schoenleini and Trichophyton rubrum.

These infections are usually quite uncomfortable and affect different parts of the body including the scalp, beard area, nails, hands, feet, groin, trunk, arms and legs. Some commonly contagious routes include touching an infected person, from damp surfaces such as shower floors, or from a pet.

When fungus infect the nail, also known as onychomycosis, the nail becomes discolored, thick, brittle, crumble, with shape distortions and dull, turning into a medical problem that alters not only nails but also social healthiness. Frequently, onychomycosis is accompanied by onycholysis, a condition when infected finger or toe nails separate from the nail bed, causing pain and a slightly foul odor. Unfortunately, the condition can get worse as nail fungus can spread to other nails or the skin, and can be accompanied by a secondary bacterial or yeast infection in the nail bed which can cause permanent damage if left untreated.

Fungi infections are commonly treated but success depends on the site of infection and on compliance with treatment. Such is the case of finger and toe nail fungus infections. They are usually difficult to treat as treatment failures and recurrences are common, seeming a never-ending problem. Furthermore, nails require between 12 to 18 months to growth, depending on age, gender, season, exercise level, diet and hereditary factors. Thus, treatment effectiveness is essential as the end result of treatment will only be seen after the nail grows back completely. Different treatments have been proposed including antifungal oral medications, nail creams, ointments and lacquers. The most typical drugs prescribed are Itraconazole, Fluconazole, Ketoconazole, Griseofulvin, and Terbinafine. These antifungal oral medications help a new nail grow free of infection, slowly replacing the infected portion of your nail but they do not immediately improve the appearance of the nail. The most common side effects of these drugs include headaches, stomach upset, skin rashes and changes in taste sensation. Additionally, due to the extended period of time required for these drug treatments, blood tests are regularly needed to check the patient's liver function during the course of therapy. In some severe infections or following recurrence, removal of the nail is prescribed. A new nail will usually grow in its place taking as long as a year to grow back completely, though it may grow with abnormal shape or appearance. In addition, it is a painful treatment which usually renders the nail susceptible to infections requiring a regular application of antibiotics with its associated drawbacks.

The problem with the previously mentioned treatment methods is that many of them have proved to be ineffective or produced considerable side effects ranging from skin rashes to liver damage or may interact with other commonly used drugs.

Photodynamic therapy (PDT) has been proposed as an enticing alternative of eliminating fungi in toenail infections because it provides an interesting mechanism of action comprising a photosensitizer which is activated by a suitable light irradiation wavelength. Such is the case of U.S. Pat. No. 6,090,788 and International Publication No WO 99/04628 disclosed by Lurie, providing a method for treating an area of skin or nail affected with a pathogen comprising the step of irradiating the area with a light beam having a wavelength absorbable by the pathogen, as the pathogen includes a light absorbing substance.

However, to avoid the complications associated to externally administer a photosensitive substance, further approaches comprise solely an irradiation step which has also shown desirous anti-bacterial and anti-microbial benefits.

One example is the application of thermal energy or radiation source for treating diseased nails without the use of a dying agent or an exogenous chromophore, disclosed in Patent Application Publication No US 2006/0212098 and International Publication No WO 2006/076506 A2 by Demetriou et al. The energy source is applied to the target area to thermally deactivate an unwanted organism such as a bacterium, mold, fungus, parasite or virus without causing substantial unwanted injury to at least one of the nail bed and the nail plate. The method further comprises the introduction of an index matching solution, such as mineral oil, glycerin, glycol or water, into a porous region of the diseased nail prior to delivering radiation.

Another example is disclosed in Patent Application No US 2009/0118721 A1 and International Publication No WO 2007/014130 A2 by Bornstein. Near infrared radiant energy in the range of 850 nm-900 nm and/or 905 nm-945 nm at suitable dosimetries is used to impair biological contaminants such as fungus, without intolerable risks and/or adverse effects to biological moieties other than a target biological contaminant. Furthermore, they propose for certain applications, laser light to be applied in two spectral wavelengths, including 870 nm and 930 nm, respectively.

However, not only near infrared light has been proposed as a treatment for fungal diseases. Patent Application No US 2006/0241729 A1 disclosed by Dawson provides a method of treating nail fungus comprising the application of ultraviolet light selected from the group of UV-C 254 nm to an affected nail, with the aim of altering the DNA of the infectious fungus on the nail bed. Another example is Patent Application No US 2007/0104664A1 disclosed by Maltezos et al., in which they present a device and method that provides ultraviolet light and heat to fingernails or toenails that are afflicted by onychomycosis. In this case, ultraviolet light is provided by an ultraviolet light emitting diode and heat is provided by a resistive heating element. Nevertheless, there are disquieting risks associated to the use of UV radiation for treating nail fungus because it is an ionizing radiation responsible of direct collagen, fiber and DNA damage. Additionally, there are some doubts that ultraviolet rays would penetrate an infected nail sufficiently to fully affect the fungus growing underneath.

Thus, there is a need to effectively treat the extremely difficult-to-treat pathogenic microbes which cause onychomycosis and dermatophytosis. It would be advantageous to provide methods of treatment to successfully deal with pathogenic microbes and overcome the drawbacks of prior art methods.

The present invention addresses this need.

### BRIEF SUMMARY AND OBJECTIVES OF THE INVENTION

It is an objective of the present invention to provide a method for treatment of skin and nail microbial infections, preferably fungi causing dermatophytosis such as Tinea barbae, Tinea capitis, Tinea cruris and Tinea unguium.

It is a further objective of the present invention to effectively target the unwanted fungal species without externally administered additives by using a light radiation source operating in the range of near infrared light wavelengths of the electromagnetic spectrum.

It is a still further objective of the present invention to provide devices emitting near infrared light wavelengths to effectively target the unwanted fungal species by irradiation, applied either by a health professional or as repeated treatment by the patient at home e.g. using a laser brush or a lamp.

Briefly stated, the present invention discloses methods and devices for treating skin and nail infections such as onychomycosis and dermatophytosis, caused by pathogenic microbes such as fungi. In a preferred embodiment, a method for treating skin and nail infections comprises irradiating the treatment site with a light radiation source operating at one or more near infrared wavelengths which are absorbed by the pathogenic microbes, or operating at 980±30 nm, 1470±40 nm and/or 1900±60 nm and combinations of these. In another preferred embodiment, a method for treating skin and nail fungus comprises irradiating the treatment site with a device emitting laser radiation of 980±30 nm wavelength. In another embodiment, a device for treating toenail fungus comprises a laser radiation source which emits a combination of radiation wavelengths at about 980±30 nm and 1470±40 nm; or 980±30 nm and 1900±60 nm; or 1470±40 nm and 1900±60 nm; or 980±30 nm, 1470±40 nm and 1900±60 nm. In another embodiment devices for treating skin and nail infections includes a laser radiation source and delivery apparatuses such as brush or toothed comb for nail and scalp infections respectively.

The above, and other objectives, features and advantages of the present invention will become apparent from the following description.

### BRIEF DESCRIPTION OF FIGURES

FIG. 1a shows a side view of one embodiment depicting a delivery apparatus to treat fungal infections in toe and finger nails.

FIG. 1b depicts a front view of delivery apparatus to treat fungal infections in toe and finger nails.

FIG. 2a depicts a side view of one embodiment of delivery apparatus to treat fungal infections in the scalp.

FIG. 2b shows a frontal view of delivery apparatus to treat fungal infections in the scalp.

### DETAILED DESCRIPTION OF PREFERRED EMBODIMENTS

The present invention provides methods and devices for disinfecting or sterilizing an infected nail or a contaminated area of the skin. In a preferred embodiment, a method to effectively destroy or inactivate infectious microbe species such as fungi, viruses, bacteria and yeast comprises applying light radiation to infected areas of the skin or nail. Firstly, the selected treatment site is cleaned with abundant water to remove any loose debris or unwanted dirt in order to allow appropriate exposure of the infected area. After nail cleaning and hydration, the nail is dried with a lint-free paper. Then, radiation from a light radiation source is applied to a selected treatment site for a desired period of time. Irradiation step is performed repeatedly, at predetermined time intervals in the range of hours to days. Light radiation source emits radiation of wavelength in the near infrared region of the electromagnetic spectrum. One objective is to deliver at least a wavelength having the ability to be absorbed by water of tissue around and/or in contact with infected cells or by the infectious microbe species. After the water surrounding and/or in contact with infected cells absorbs radiation, it generates hyperthermia and transfers thermal energy to the infectious microbes in order to destroy or inactivate them by thermal energy. In another situation, infectious microbes or treated tissue can have an endogenously light absorbing substance. In this case, the delivered light radiation wavelength absorbed by infectious microbes or treated tissue can produce a cytotoxic effect generating reactive singlet oxygen due to the interaction between radiation and chromophore. In any case the aim is to kill, destroy or inactivate pathogenic microbes by impairing vital organism functions, reproduction and/or others. Eventually, a partial killing, destruction or inactivation of pathogenic microbes can provoke an immune response by the host organism which leads to further healing of the treatment site.

Wavelengths at 980 nm, 1470 nm 1900 nm are effectively absorbed by water and blood thus temperature is raised without needing the administration of exogenous additives. Due to the effective energy absorption, the temperature is raised only to impair microbes' vital organism functions without damaging surrounding healthy tissue. Suitable irradiation parameters such as time, energy and power density are adjusted according to patient's disease condition. Preferably, irradiation parameters are adjusted according to the radiation source and spot size to raise the temperature up to about 40-45° C., in order to avoid damaging host tissue. In another embodiment, light radiation sources include one or more radiation wavelengths absorbed by any endogenously light absorbing substance present in the microbes or treated tissue.

In preferred aspects of the invention, the described treatment methods with light irradiation can be applied to a variety of tasks including:
the destruction of disease-related microbes such as fungi and yeast in the nail in order to treat or prevent chronic onychomycosis and the like;
the destruction of disease-related microbes such as fungi and yeast in a contaminated area of the skin in order to treat or prevent skin-borne diseases including chronic dermatophytosis and the like.

The method employed to destroy or inactivate infectious microbes with light radiation effectively heals chronic fungal/bacterial diseases in the nail or skin. The preferred light radiation source has a range of wavelengths in the near infrared region of the electromagnetic spectrum, specifically operating at 980±30 nm, 1470±40 nm and/or 1900±60 nm.

Using these different light wavelength ranges allows achieving different penetration depths through the nail thickness with different water and blood absorption characteristics, providing an enhancement of microbial inactivation effect reaching simultaneously different difficult-to-treat areas during the same treatment.

In a preferred embodiment, the present invention provides devices comprising a light radiation source and a delivery apparatus, to effectively destroy or inactivate infectious microbe species such as fungi, viruses, bacteria and yeast. Light radiation source emits near infrared light wavelengths or suitable wavelengths to activate endogenously light absorbing substances present in the microbes or treated tissue. Light radiation sources include coherent and incoherent radiation sources such as laser radiation source, light emitting diodes source and lamp radiation source (incandescent, xenon arc and metal halide lamps).

In another embodiment, light sources emit pulsed or continuous laser radiation of wavelength in the near infrared region of the electromagnetic spectrum, specifically operating at 980±30 nm, 1470±40 nm and/or 1900±60 nm. In another embodiment, the near infrared wavelength source emits a combination of laser radiation wavelengths in the ranges of 1) 980±30 nm and 1470±40 nm, 2) 980±30 nm and 1900±60 nm, 3) 1470±40 nm and 1900±60 nm, or 4) 980±30 nm, 1470±40 nm and 1900±60 nm; in a simultaneous or an alternating sequence, delivering radiation to different absorbing targets in the same treatment site. In any case, laser radiation wavelengths are chosen to provide adequate tissue penetration with sufficient energy to elevate the temperature or directly photo-destroy of the tissue around and/or in contact with infected cells, in order to destroy or inactivate infectious microbe species.

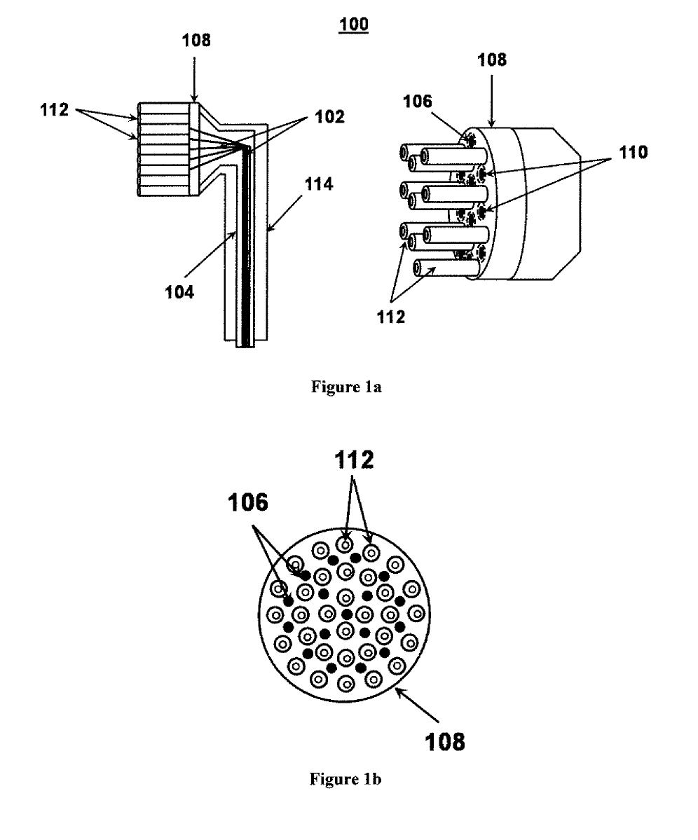

In one embodiment, laser radiation energy is distributed with the aid of a delivery apparatus, such as a brush or lamp. Laser irradiation may be applied either by a health professional or as repeated treatment by the patient at home. One preferred delivery apparatus is like the toothbrush disclosed in U.S. Pat. No. 5,658,148 and U.S. Pat. No. 6,056,548 and incorporated by reference herein. In another embodiment, a delivery apparatus to treat fungal infections in toe and finger nails is depicted in FIG. 1. The brush 100, preferably a plastic brush, contains one or more optical fibers 102 which deliver laser radiation, placed inside conduit 104. Optical fiber tip 106 may be included in brush head 108 and emits laser radiation through openings 110 in brush head 108, at the end of hollow bristles 112 inserted in brush head 108 or in both places. In order to easily operate brush 100, it is fixed to handle 114. Brush 100 is removable from handle 114 so different brush shapes and sizes containing different bristle diameters may be used depending on the treatment site and application.

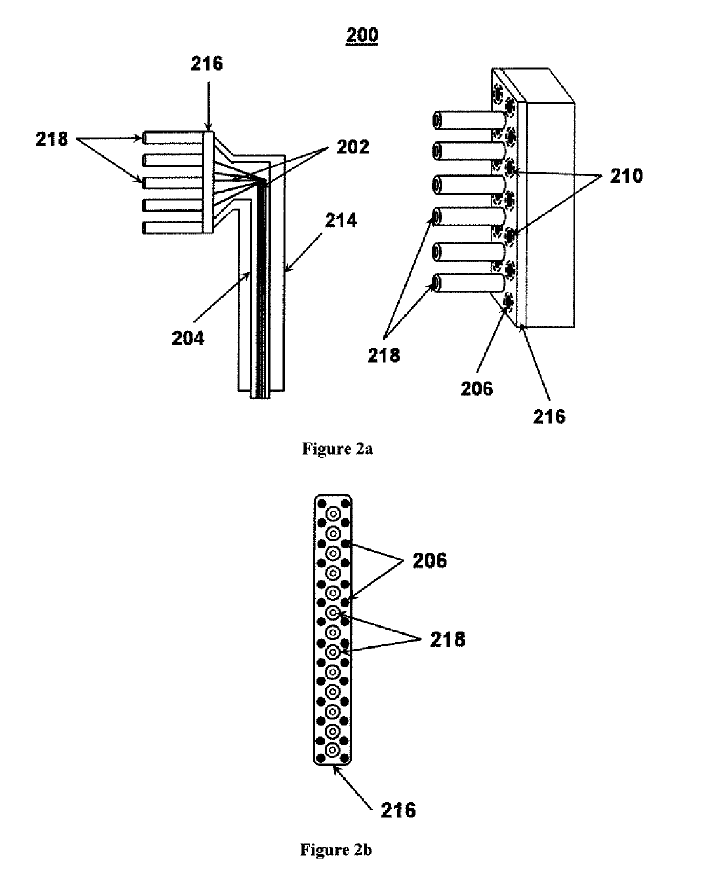

In another embodiment a delivery apparatus to treat fungal infections in the scalp is depicted in FIG. 2. The delivery apparatus is a brush like the one depicted in previous embodiment but with a size that allows effective laser radiation delivery to the treatment site. The delivery apparatus comprises a toothed comb 200, preferably made of plastic, containing one or more optical fibers 202 which deliver laser radiation, placed inside conduit 204. Optical fiber tip 206 may be included in the edge of the comb 216 and emits laser radiation through openings 210 in the edge of the comb 216, at the end of hollow comb's teeth 218 inserted in the edge of the comb 216 or in both places. In order to easily operate comb 200, it is fixed to handle 214. Comb 200 is removable from handle 214 so different comb shapes and sizes containing different comb's teeth diameters may be used depending on the treatment site.

The present invention is further illustrated by the following examples, but is not limited thereby.

### Example 1

### Fungal Reduction in Contaminated Toenail

A health professional can treat onychomycosis in various stages, or patient can self-apply the treatment for prevention of diseases arising from fungi or yeast in the toenail. The toenail to be treated is “polished” with the aid of emery board, a nail file or any other means in order to reduce the nail thickness. Next, it is cleaned with abundant water and then dried with a lint-free paper. The health professional would then administer radiation for a prescribed duration, power or energy density and pulse frequency, depending of the characteristics of the nail and using a device which comprises a laser radiation source and delivery apparatus depicted in FIG. 1. As an example, for a toenail with an area of about 2.5 cm², irradiation parameters include a power density of 400 mW/cm² (which is about 3 times the intensity of the IRA radiation from the sun) applied during 5 minutes. In this way, the temperature is raised sufficiently to impair, destroy or inactivate pathogenic microbes without irreversibly damaging the treated toenail. The infected site is irradiated with the laser source operating at a wavelength of 980 nm with a prescribed irradiation frequency of about twice a day to effectively destroy harmful fungi in the toenail.

Alternatively, in order to irradiate different tissue depths, the infected site is irradiated with a laser source operating in an alternate mariner, interspersing the 980 nm and 1470 nm laser wavelengths. To merely prevent recurrences, a less frequent irradiation might be proposed of once every day, once every other day, or even once a week or month.

Additionally, the patient can also self-apply radiation with the aid of a diode lamp, emitting radiation of 980 nm and/or 1470 nm wavelength. In these cases, supplementary care has to be taken by training the patient on the use of the lamp and appropriately setting the lamp's security parameters e.g. establishing a maximum time of treatment. In addition, protective devices covering the tissue surrounding the area to be treated should be used, to avoid potential skin damage beyond treatment site due to photochemical reactions on skin, akin to sun burn or other.

### Example 2

### Microbial Reduction in Contaminated Area of the Skin

A patient self-applies the therapy for treatment or prevention of diseases arising from fungi or yeast in the skin such as Tinea capitis. First a health professional assesses the area to be treated and depending on the nature and characteristics of the infection he prescribes irradiation parameters to be used for treatment. The patient would then administer radiation for a prescribed exposing duration, energy or power density and pulse frequency, with the aid a laser device operating at 980 nm with delivery apparatus 200. A prescribed irradiation frequency of about twice a day would effectively destroy harmful fungi in the scalp. Merely to prevent recurrences a less frequent activation might be proposed of once every day, once every other day, or even once a week or month.

Having described preferred embodiments of the invention, it is to be understood that the invention is not limited to the precise embodiments, and that various changes and modifications may be effected therein by those skilled in the art without departing from the scope or spirit of the invention as defined in the appended claims.

What is claimed is:

1. A method for the treatment of nail/skin fungal disease comprising the steps of:
a. preparing a selected treatment site by superficial cleansing with abundant water and removing any loose debris;
b. selecting a near infrared wavelength source, operating at about 980±30 nm, 1470±40 nm, 1900±60 nm and combinations of them;
c. irradiating said selected treatment site at least twice for a desired period of time separated by a predetermined delay.

2. The method according to claim 1, wherein said irradiating step is applied repeatedly at predetermined intervals.

3. The method according to claim 2, wherein said predetermined intervals range for irradiating are from a period of several hours to several days.

4. The method according to claim 1 wherein said irradiating step uses preselected wavelengths, at either 980±30 nm, 1470±40 nm, 1900±60 nm and combinations of them.

6. The method according to claim 1 wherein human nail/skin microbial infections are being treated.

7. The method according to claim 6, wherein human nail/skin microbial infections treated include dermatophytosis including Tinea barbae, Tinea capitis, Tinea cruris and Tinea unguium.

8. The method according to claim 7, wherein human nail/skin microbial infections are caused by dermatophytes belonging to the Epidermophyton, Microsporum and Trichophyton genera, including Epidermophyton floccosum, Microsporum canis, Microsporum audouinii, Trichophyton interdigitale/mentagrophytes, Trichophyton tonsurans, Trichophyton schoenleini and Trichophyton rubrum.

9. A device for the treatment of nail/skin fungal disease comprising:
a. a light radiation source operating at near infrared light wavelengths; and
b. a light delivery apparatus containing at least one optical fiber and a conduit.

10. The device according to claim 9, wherein said light radiation source includes coherent and incoherent radiation sources selected from a group consisting of laser radiation sources, light emitting diode sources, lamp radiation sources, and combinations of them.

11. The device according to claim 9, wherein said light radiation source operates at 980±30 nm, 1470±40 nm, 1900±60 nm and combinations of them.

12. The device according to claim 9, wherein said delivery apparatus further comprising removable handle, hollow bristles and teeth.
